(19) United States
(12) Patent Application Publication
(10) Pub. No.: US 2016/0228075 A1
(43) Pub. Date: Aug. 11, 2016

(54) IMAGE PROCESSING DEVICE, METHOD AND RECORDING MEDIUM

(71) Applicant: FUJIFILM Corporation, Tokyo (JP)

(72) Inventor: Yoshiro KITAMURA, Tokyo (JP)

(73) Assignee: FUJIFILM Corporation, Tokyo (JP)

(21) Appl. No.: 15/133,908

(22) Filed: Apr. 20, 2016

Related U.S. Application Data
(63) Continuation of application No. PCT/JP2014/005372, filed on Oct. 22, 2014.

(30) Foreign Application Priority Data
Oct. 25, 2013 (JP) 2013-221930

(51) Int. Cl. A61B 6/12; A61B 6/03; A61B 1/00; A61B 6/00

(52) U.S. Cl. CPC A61B 6/12; A61B 6/5235; A61B 6/463; A61B 6/032; A61B 1/00009

(57) ABSTRACT

Employing a first image and a second image that represent a subject in different phases, based on a first insertion position and a first tip position of the first image and deformation information for deforming the first image so as to be aligned with the second image, a second insertion position of the second image corresponding to the first insertion position and a second tip position are specified such that a direction corresponding to a first insertion direction from the first insertion position toward the first tip position becomes a second insertion direction from the second insertion position toward the second tip position, and a second observation image obtained by visualizing the inside of the subject in a phase corresponding to the second image with the second tip position as a viewpoint is generated. Thereby, observation images of different phases as viewed through a virtual rigid surgical device are generated.

### CROSS REFERENCE TO RELATED APPLICATIONS

The present application is a Continuation of PCT International Application No. PCT/JP2014/005372 filed on Oct. 22, 2014, which claims priority under 35 U.S.C. §119(a) to Japanese Patent Application No. 2013-221930 filed on Oct. 25, 2013. Each of the above applications is hereby expressly incorporated by reference in its entirety, into the present application.

### BACKGROUND OF THE INVENTION

1. Field of the Invention

The present invention relates to an image processing device, a method, and a non transitory computer readable recording medium, in which an image processing program is stored, and in particular, to an image processing device, a method, and a non transitory computer readable recording medium, in which an image processing program is stored which generate an observation image obtained by visualizing the inside of a subject from a three-dimensional image data indicating the inside of the subject.

2. Description of the Related Art

In recent years, with the advancement of imaging devices (modalities), such as multi detector-row computed tomography (MDCT), high-quality three-dimensional image data has been able to be acquired, and in image diagnosis using these kinds of image data, not only a high-definition cross-sectional image is used, but also a virtual or pseudo three-dimensional image of a subject is used.

With the advancement of the above-described technique, many tumors, such as cancers, have been found in a comparatively early period, such as an early stage. In a comparatively early period, since the cancers are small in size, and a risk of metastatis is low, treatment using shrinking surgery for removing a region necessary and sufficient for curing the cancer has been actively used. Endoscopic surgery which is one shrinking surgery has a small burden on the body; however, a technical difficulty in carrying out desired treatment so as not to damage nearby organs or blood vessels within a limited field of view under an endoscope is high. In order to support endoscopic surgery, a technique which extracts an organ and the like from three-dimensional image data using an image recognition technique and generates and displays a virtual and pseudo three-dimensional image from a three-dimensional image with an organ identified has been suggested, and is used for planning and simulation before surgery or navigation during surgery.

JP2012-187161A discloses a technique which acquires two three-dimensional images obtained by imaging a subject in different postures, such as a supine position and a prone position, generates a virtual endoscopic image from an arbitrary viewpoint from one of the two three-dimensional image, generates a virtual endoscopic image from the other image with a point corresponding to the viewpoint set for one image as a viewpoint, and simultaneously displays the two generated images on a display screen. JP2008-005923A discloses a technique which acquires an ultrasound endoscopic image obtained by imaging a subject in a left lateral decubitus position and a three-dimensional image obtained by imaging the subject in a supine position, corrects the three-dimensional image such that an organ of the acquired three-dimensional image becomes an organ in a case where the subject is in the left lateral decubitus position, and generates and displays an image of a section in the position and direction corresponding to the ultrasound endoscopic image from the corrected three-dimensional image. JP2013-000398A discloses a technique which displays an ultrasound image and an image of a section corresponding to a magnetic resonance image (MR image) deformed so as to be aligned with the ultrasound image in a comparable manner.

### SUMMARY OF THE INVENTION

On the other hand, unlike a soft endoscope shown in JP2012-187161A and JP2008-005923A in which an insertion portion is inserted into the subject, in order to image an observation target through a curved path inside a celom, in a medical instrument, such as a rigid endoscope device having an elongated rigid insertion portion inserted into the subject, since the inflexible (unbending) elongated rigid insertion portion is arranged from an insertion port of the subject toward a tip portion of the endoscope device, an insertion direction accessible from the insertion port is limited. For this reason, in surgery using the rigid endoscope device, for observation or treatment of the inside of the subject, when determining the tip position or posture (direction) of the endoscope device, the relative relationship between the insertion port and the tip position including the insertion direction of the insertion port of the medical instrument, such as a rigid endoscope device, should be appropriately determined.

There is a case where there are a plurality of phases of respiration or pulsation causing deformation of an anatomical structure inside a subject in a period during which a rigid medical instrument, such as an endoscope device, is arranged inside the subject, for example, during surgery, or the like. In this case, it is considered that it is preferable to confirm whether or not the tip position and the insertion direction of the endoscope device are set at an appropriate position and distance with respect to a desired treatment part in different phases of a three-dimensional image representing a subject. For this reason, it is preferable to confirm whether or not the tip position and the insertion direction of the endoscope device are set in both observation images at an appropriate position and distance with respect to a desired treatment part not only by generating and displaying an observation image, such as a virtual endoscopic image, generated based on a set viewpoint of a virtual endoscope device and an imaging direction in the three-dimensional image of one phase but also by generating and displaying an observation image, such as a virtual endoscopic image, based on a viewpoint of the virtual endoscope device inserted from a corresponding insertion port and an imaging direction in the three-dimensional image of the other phase.

However, according to the techniques described in JP2012-187161A, JP2008-005923A, and JP2013-000398A, although two virtual endoscopic images can be generated from the two three-dimensional images with mutually corresponding positions as viewpoints, in the generated virtual endoscopic images, the relative relationship between the insertion port and the tip position is not made correspondent in a case where a rigid endoscope device is used as a virtual endoscope device.

The invention has been accomplished in consideration of the above-described situation, and an object of the invention is to provide an image processing device, a method, and a program which, in three-dimensional images representing the inside of a subject in different phases, generates a first observation image in one phase based on a viewpoint of a virtual endoscope device set in the three-dimensional image corresponding to one phase and an imaging direction, and in the three-dimensional image corresponding to a different phase, generates an observation image in the different phase while making the relative relationship between an insertion port from which a medical instrument having a rigid insertion portion, such as a virtual endoscope device, is inserted into a subject and a tip position correspondent.

In order to solve the above-described problem, an image processing device according to the invention comprises a three-dimensional image acquisition unit which acquires a first image and a second image respectively representing the inside of a subject in different phases as three-dimensional images captured using a medical imaging device, a deformation information acquisition unit which acquires deformation information for deforming the first image such that corresponding positions of the first image and the second image are aligned with each other, an observation condition determination unit which acquires a first insertion position to be the insertion position of a surgical instrument having an elongated rigid insertion portion inserted into the body of the subject and a first tip position to be the position of a tip portion of the surgical instrument from the first image as a first observation condition, based on the first observation condition and the deformation information, specifies a second insertion position to be the position on the second image corresponding to the first insertion position and specifies a second tip position such that a direction corresponding to a first insertion direction from the first insertion position toward the first tip position becomes a second insertion direction from the second insertion position toward the second tip position to be the position of the tip portion of the surgical instrument in the second image, and determines the second insertion position and the second tip position as a second observation condition, and an image generation unit which generates a second observation image obtained by visualizing the inside of the subject from the second tip position from a deformed first image obtained by deforming the first image based on the deformation information or the second image based on the second observation condition with the second tip position as a viewpoint.

A method of operating an image processing device according to the invention comprises a three-dimensional image acquisition step of acquiring a first image and a second image respectively representing the inside of a subject in different phases as three-dimensional images captured using a medical imaging device, a deformation information acquisition step of acquiring deformation information for deforming the first image such that corresponding positions of the first image and the second image are aligned with each other, an observation condition determination step of acquiring a first insertion position to be the insertion position of a surgical instrument having an elongated rigid insertion portion inserted into the body of the subject and a first tip position to be the position of a tip portion of the surgical instrument from the first image as a first observation condition, based on the first observation condition and the deformation information, specifying a second insertion position to be the position on the second image corresponding to the first insertion position and specifying a second tip position such that a direction corresponding to a first insertion direction from the first insertion position toward the first tip position becomes a second insertion direction from the second insertion position toward the second tip position to be the position of the tip portion of the surgical instrument in the second image, and determining the second insertion position and the second tip position as a second observation condition, and an image generation step of generating a second observation image obtained by visualizing the inside of the subject from the second tip position from a deformed first image obtained by deforming the first image based on the deformation information or the second image based on the second observation condition with the second tip position as a viewpoint.

An image processing program according to the invention causes a computer to execute the above-described method.

“The first image and the second image respectively representing the inside of the subject in different phases” may be images with different deformation states of the inside of the subject. For example, the first image and the second image may respectively represent the subject in an expiration phase and an inspiration phase, or the first image and the second image may respectively represent the subject in different pulsation phases. The first image and the second image may represent the inside of the subject in different postures.

As “the surgical instrument having the elongated rigid insertion portion inserted into the body of the subject” is, for example, a rigid endoscope device in which a camera is arranged at the tip of a rigid elongated cylindrical body portion, a rigid treatment tool in which a treatment tool, such as a scalpel or a needle, is arranged at the tip of a rigid elongated cylindrical body portion, or the like is considered. The rigid insertion portion includes an insertion portion in which a flexible portion is provided at the tip of an unbending body portion.

“The tip portion of the surgical instrument” means a portion where a camera or a treatment tool for performing desired observation or treatment is arranged in the rigid insertion portion inserted into the inside of the subject, and may not necessarily be the tip of the surgical instrument.

In the image processing device according to the invention, it is preferable that the surgical instrument is an endoscope device, the observation condition determination unit specifies the second imaging direction such that the relative relationship between the first insertion direction in the first image and a first imaging direction to be the imaging direction of the endoscope device becomes equal to the relative relationship between the second insertion direction in the second image and a second imaging direction to be the imaging direction of the endoscope device, and the image generation unit generates the second observation image by visualizing the inside of the subject in the second imaging direction from the second tip position.

In the image processing device according to the invention, the observation condition determination unit may specify the second tip position such that the distance between the first insertion position and the first tip position becomes equal to the distance between the second insertion position and the second tip position, and may determine the second insertion position and the second tip position as the second observation condition. Alternatively, the observation condition determination unit may specify the position on the second image corresponding to the first tip position as the second tip position, and may determine the second insertion position and the second tip position as the second observation condition.

In the image processing device according to the invention, it is preferable that the observation condition determination unit specifies the second insertion direction such that the direction corresponding to the first insertion direction becomes the second insertion direction by specifying the second insertion direction such that the angle between the direction of a predetermined landmark included in the first image and the first insertion direction becomes equal to the angle between the direction of the predetermined landmark included in the second image and the second insertion direction.

“The direction of the predetermined landmark” is the direction which is specified by the predetermined landmark included in the three-dimensional image, and can be, for example, the direction normal to the body surface of the subject at the insertion position where the surgical instrument is inserted. An arbitrary portion can be used as a landmark as long as the portion is an identifiable feature portion included in the three-dimensional image. It is preferable to use a landmark with less fluctuation in the direction of the landmark according to the phase. For example, a backbone can be used as a landmark, and in this case, the position of an N-th vertebra can be used as a landmark. (The center coordinates) of an organ, such as spleen or kidney, may be used as a landmark. “The direction which is specified by the landmark” may be any direction as long as the direction may be a direction which is specified by the landmark. For example, if a landmark has a flat shape, a direction normal to the flat shape may be used. If a landmark has a longitudinal shape, the direction which is specified by the landmark may be the direction of the axis of the longitudinal shape. “The direction which is specified by the landmark” may be a direction which is specified by a plurality of landmarks. In this case, a direction from a landmark, such as a center point of one structure, toward a landmark, such as a center point of another landmark may be used.

In the image processing device according to the invention, it is preferable that the observation condition determination unit acquires a plurality of first observation conditions from the first image and determines a plurality of second observation conditions corresponding to the plurality of first observation conditions based on the plurality of first observation conditions and the deformation information.

In the image processing device according to the invention, it is preferable that a determination unit which determines whether or not a line segment connecting the second insertion position and the second tip position is equal to or less than a predetermined distance from an anatomical structure included in the second image.

In the image processing device, the method, and the program of the invention, the first image and the second image respectively representing the inside of the subject in different phases as the three-dimensional images captured using the medical imaging device are acquired, the deformation information for deforming the first image such that the corresponding positions of the first image and the second image are aligned with each other is acquired, the first insertion position to be the insertion position of the surgical instrument having the elongated rigid insertion portion inserted into the body of the subject and the first tip position to be the position of the tip portion of the surgical instrument are acquired from the first image as the first observation condition, based on the first observation condition and the deformation information, the second insertion position to be the position on the second image corresponding to the first insertion position is specified and the second tip position such that the direction corresponding to the first insertion direction from the first insertion position toward the first tip position becomes the second insertion direction from the second insertion position toward the second tip position to be the position of the tip portion of the surgical instrument in the second image is specified, and the second insertion position and the second tip position are determined as the second observation condition. The second observation image obtained by visualizing the inside of the subject from the second tip position is generated from the deformed first image obtained by deforming the first image based on the deformation information or the second image based on the second observation condition with the second tip position as a viewpoint.

For this reason, in the second image of a phase different from the first image, the tip position (second tip position) of the virtual medical instrument in the second image is determined corresponding to the insertion position (first insertion position) and the insertion direction (first insertion direction) of the virtual medical instrument in the first image and the insertion position (second insertion position) and the insertion direction (second insertion direction) of the virtual medical instrument in the second image, whereby it is possible to generate the second observation image obtained by visualizing the inside of the subject with the second tip position as a viewpoint. For this reason, even in a case where there are a plurality of phases of respiration or pulsation causing deformation of an anatomical structure inside the subject in a period during which the medical instrument having the rigid insertion portion is arranged inside the subject, for example, during surgery, or the like, when carrying out treatment or observation of the inside of the subject in a phase corresponding to the second image by observing the generated second observation image, it is possible to provide useful information for easily and accurately determining whether or not the insertion position, the tip position, and the insertion direction with respect to the inside of the subject are appropriate.

### BRIEF DESCRIPTION OF THE DRAWINGS

FIG. 1 is a block diagram showing an image processing device according to an embodiment of the invention.

FIG. 2 is a diagram (first view) illustrating a screen for setting a tip position and an insertion direction of an endoscope device in a first image.

FIG. 3 is a diagram (second view) illustrating a screen for setting the tip position and the insertion direction of the endoscope device in the first image.

FIG. 4 is a diagram illustrating a method of specifying an insertion position, an insertion direction, and a tip position of an endoscope device in a second image.

FIG. 5 is a flowchart showing an operation procedure of the image processing device according to the embodiment of the invention.

### DESCRIPTION OF THE PREFERRED EMBODIMENTS

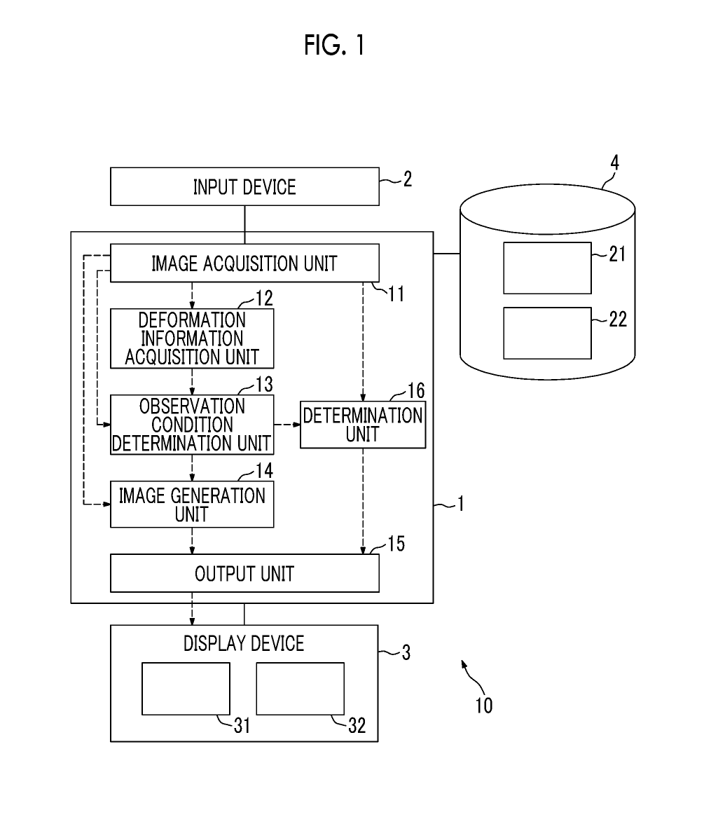

Hereinafter, an embodiment of the invention will be described in detail referring to the drawings. FIG. 1 shows an image processing workstation 10 including an image processing device 1 according to an embodiment of the invention.

The image processing workstation 10 is a computer which performs image processing (including image analysis) on medical image data acquired from a modality or an image storage server (not shown) in response to a request from a reader, and displays a generated image, and includes an image processing device 1 which is a computer body including a CPU, an input/output interface, a communication interface, a data bus, and the like, and known hardware configurations, such as an input device 2 (mouse, keyboard, and the like), a display device 3 (display monitor), and a storage device 4 (main storage device, auxiliary storage device). The image processing workstation 10 has a known operating system, various kinds of application software, and the like installed thereon, and has an application for executing image processing of the invention installed thereon. These kinds of software may be installed from recording mediums, such as CD-ROM, or may be downloaded from a storage device, such as a server, connected through a network, such as the Internet, and installed.

As shown in FIG. 1, the image processing device 1 according to this embodiment includes an image acquisition unit 11, a deformation information acquisition unit 12, an observation condition determination unit 13, an image generation unit 14, an output unit 15, and a determination unit 16. The functions of the respective units of the image processing device 1 are realized by the image processing device 1 which executes the program (image processing application) installed from a recording medium, such as a CD-ROM.

The image acquisition unit 11 acquires a first image 21 and a second image 22 from the storage device 4. The first image 21 and the second image 22 are respectively three-dimensional image data indicating the inside of a subject imaged using a CT device. The image acquisition unit 11 may acquire the first image 21 and the second image 22 simultaneously, or may acquire one of the first image 21 and the second image 22 and then may acquire the other image.

In this embodiment, the first image 21 and the second image 22 are data obtained by imaging the abdomen of the subject (human body) in different respiration phases. The first image 21 is an image captured in an expiration phase, and the second image 22 is an image captured in an inspiration phase. Both images represent the inside of a celom of a person, but the respiration phases at the time of imaging are different; thus, an organ shape is deformed in both images.

The invention is not limited to this embodiment, and the first image 21 and the second image 22 may be any images as long as the images are three-dimensional image data with different deformation states of the inside of the subject obtained by imaging the inside of the subject. For example, as the second image 22, a CT image, an MR image, a three-dimensional ultrasound image, a positron emission tomography (PET) image, or the like can be applied. A modality for use in tomographic imaging may be any of CT, MRI, an ultrasound imaging device, or the like as long as a three-dimensional image can be captured. As a combination of the first image 21 and the second image 22, various combinations are considered. For example, the first image 21 and the second image 22 may be data imaged in different imaging postures. Alternatively, the first image 21 and the second image 22 may be a plurality of images respectively representing the subject in different pulsation phases.

The deformation information acquisition unit 12 acquires deformation information for deforming the first image such that corresponding positions of the first image 21 and the second image 22 are aligned with each other.

Each pixel of the first image 21 corresponds to each pixel of the second image 22 corresponding to each pixel of the first image 21 by setting a deformation amount in each pixel of the first image 21 and maximizing (minimizing) a predetermined function representing similarity between an image obtained by deforming each pixel of the first image 21 based on each deformation amount while gradually changing each deformation amount and the second image 22, and the deformation amount of each pixel for aligning the first image 21 with the second image 22 is acquired. A function which defines the deformation amount of each pixel of the first image 21 is acquired as deformation information.

A nonrigid registration method is a method which calculates the deformation amount of each pixel of one image for aligning two images with each other by maximizing (minimizing) a predetermined function which moves each pixel of one image based on each deformation amount to determine similarity between two images. In this embodiment, for example, various known methods, such as D. Rueckert, L. I. Sonoda, C. Hayes, et al., “Nonrigid Registration Using Free-Form Deformations: Application to Breast MR Images”, IEEE transactions on Medical Imaging, 1999, Vol. 18, No. 8, pp. 712-721, can be applied as long as a nonrigid registration method can aligns two images with each other.

The observation condition determination unit 13 acquires the coordinates of a first insertion position Q_{A1} to be the center position of a virtual insertion port of a virtual endoscope device M1 (virtual rigid endoscope device) as a surgical instrument having an elongated rigid insertion portion inserted into the body of the subject, the coordinates of a first tip position P_{A1} to be a position where a camera of the virtual endoscope device M1 is arranged, a first insertion direction (first insertion vector V_{A1}) to be a direction from the first insertion position Q_{A1} toward the first tip position P_{A1}, and a first imaging direction to be a relative camera posture with respect to the first insertion vector V_{A1} from the first image 21 as a first observation condition.

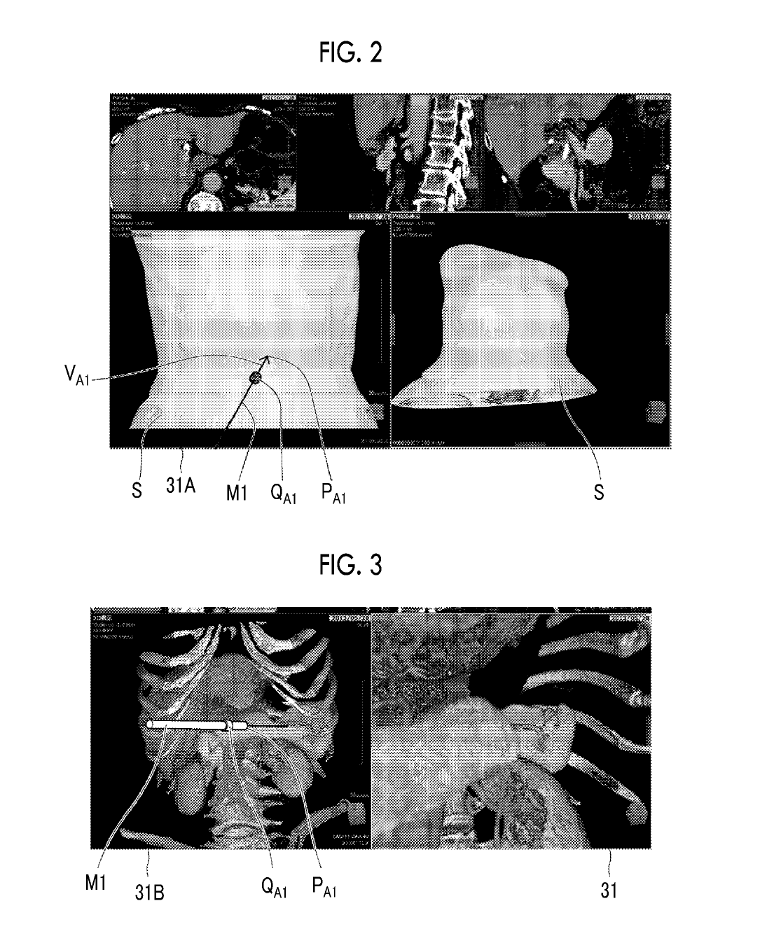

FIGS. 2 and 3 are diagrams illustrating a screen for setting the insertion position Q_{A1} and the tip position P_{A1} of the virtual endoscope device M1 in the first image 21.

As shown in FIGS. 2 and 3, if an instruction to generate a pseudo three-dimensional image from the first image 21 and an instruction to display a pseudo three-dimensional image, such as a volume rendering method, are received from a user through the input device 2, such as a mouse, the image generation unit 14 generates an image according to the generation instruction from the first image 21, and the output unit 15 displays the image generated from the first image 21 on a display screen according to desired display parameters. Reference numeral 31A of FIG. 2 is an example where display parameters are set so as to visualize a body surface S of a subject and the subject is displayed in a pseudo three-dimensional manner, and reference numeral 31B of FIG. 3 is an example where the body surface of the subject is made transparent, display parameters are set so as to visualize the inside of the subject, and the subject is displayed in a pseudo three-dimensional manner. In FIG. 3, the tip position P_{A1} of the virtual endoscope device M1 is set as a camera position (viewpoint of virtual endoscopic image) arranged as shown in 31B of FIG. 3, and a first observation image 31 which is a virtual endoscope generated so as to visualize the inside of the subject based on the set camera posture (imaging direction) of the virtual endoscope device M1 is shown.

The input device 2 receives the camera position of the virtual endoscope device M1 in the first image 21 and the camera posture of the virtual endoscope device M1 in the first image 21 based on the user input on the display screen. Then, based on information received by the input device 2, the observation condition determination unit 13 acquires the camera position of the virtual endoscope device M1 as the first tip position P_{A1}, and acquires the camera posture of the virtual endoscope device M1 as the first insertion direction (first insertion vector V_{A1}) to be a direction in which the rigid endoscope device is inserted into the inside of the subject. The observation condition determination unit 13 acquires an intersection, at which a line segment parallel to the first insertion vector V_{A1} and passing through the first tip position P_{A1} intersects the body surface S of the subject, as the coordinates of the first insertion position Q_{A1}, at which the virtual endoscope device M1 is inserted into the inside of the subject. The observation condition determination unit 13 calculates the distance D_{A1} between the first tip position P_{A1} and the first insertion position Q_{A1}.

In the virtual endoscope device M1 of this embodiment, it is assumed that the first insertion vector V_{A1} is parallel to the optical axis of the camera of the virtual endoscope device M1, and the first insertion vector V_{A1} can be regarded as the camera posture (first imaging direction) of the virtual endoscope device M1. In the first observation condition, it is assumed that other parameters necessary for generating an observation image from a three-dimensional image are set in advance according to the image angle, the focal distance, or the like of the virtual endoscope device M1, and the relative angle of the first imaging direction with respect to the first insertion direction is set in advance.

The observation condition determination unit 13 may use an arbitrary method which can acquire the first observation condition. For example, in regard to the first observation condition, a first observation condition set by manual input of the user may be acquired like the above-described example, a region to be processed of the first image 21 may be acquired and analyzed and the tip position, the insertion port, and the insertion direction of the endoscope device capable of imaging the region to be processed may be set automatically.

If the first observation condition is acquired, the observation condition determination unit 13 specifies the coordinates on the second image 22 corresponding to the coordinates of the first insertion position Q_{A1} as the coordinates of a second insertion position Q_{A2} based on the deformation information for deforming the first image 21 so as to correspond to the second image 22.

The observation condition determination unit 13 specifies a second tip position P_{A2} such that a direction corresponding to the first insertion vector V_{A1} from the first insertion position Q_{A1} toward the first tip position P_{A1} becomes a second insertion vector V_{A2} from the second insertion position Q_{A2} toward the second tip position P_{A2} to be the position of the tip portion of the surgical instrument in the second image 22, and the distance D_{A1} between the first insertion position Q_{A1} and the first tip position P_{A1} becomes equal to the distance D_{A2} between the second insertion position Q_{A2} and the second tip position P_{A2}, and determines the second insertion position Q_{A2} and the second tip position P_{A2} as a second observation condition.

The observation condition determination unit 13 specifies the relative relationship between the first insertion direction and the first imaging direction to be the imaging direction of the endoscope device, and specifies the second imaging direction such that the relative relationship between the second insertion direction and the second imaging direction to be the imaging direction of the endoscope device in the second image 22 become equal to the relative relationship between the first insertion direction and the first imaging direction to be the imaging direction of the endoscope device. Since the first insertion vector V_{A1} is parallel to the optical axis of the camera of the virtual endoscope device M1, and the first insertion vector V_{A1} is regarded as the camera posture (first imaging direction) of the virtual endoscope device M1, the observation condition determination unit 13 determines the second insertion vector V_{A2} as the camera posture (second imaging direction) of the virtual endoscope device M1 in correspondence thereto. In a case where the camera posture is at a predetermined angle, such as 45 degrees or 90 degrees, with respect to the axial direction (longitudinal direction) of the rigid insertion portion of the virtual endoscope device M1, for example, the observation condition determination unit 13 acquires the angle between the first insertion vector V_{A1} and the first imaging vector (first imaging direction) to be the imaging direction of the endoscope device in the first image 21, and determines the second imaging direction such that the angle between the second insertion vector V_{A2} and the second imaging vector (second imaging direction) in the second image becomes equal to the angle between the first insertion vector V_{A1} and the first imaging vector.

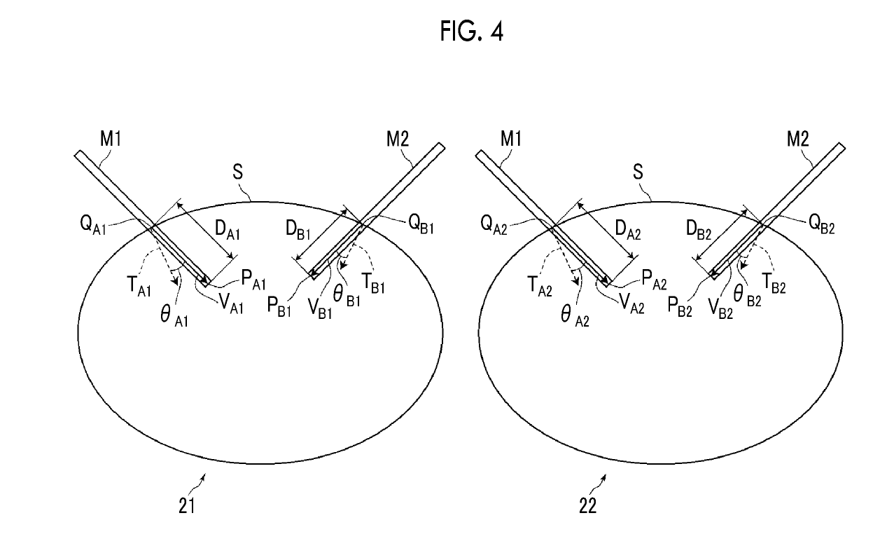

FIG. 4 is a diagram illustrating a method of specifying the insertion position (second insertion position Q_{A2}), the insertion direction (second insertion vector V_{A2}), and the tip position (second tip position P_{A2}) of the virtual endoscope device M1 in the second image 22. FIG. 4 is a diagram for illustration, and the size, position, angle, and the like of each unit is different from an actual unit. If the second insertion position Q_{A2} corresponding to the first insertion position Q_{A1} is acquired, the observation condition determination unit 13 acquires a normal vector T_{A1} of the body surface S of the subject at the first insertion position Q_{A1} from the first image 21, and acquires a normal vector T_{A2} of the body surface S of the subject at the second insertion position Q_{A2} from the second image 22.

Next, the observation condition determination unit 13 determines the second insertion vector V_{A2} such that the angle θ_{A2} between the second insertion vector V_{A2} and the normal vector T_{A2} in the second image 22 becomes equal to the angle θ_{A1} between the first insertion vector V_{A1} and the normal vector T_{A1} in the first image 21. The observation condition determination unit 13 determines the second insertion vector V_{A2} such that the inner product of the insertion vector V_{A2} from Q_{A2} toward P_{A2} and the normal vector T_{A2} becomes equal to the inner product of the first insertion vector V_{A1} and the normal vector T_{A1}.

The observation condition determination unit 13 may determine the second insertion vector V_{A2} such that the angle between a vector parallel to the direction of another predetermined landmark of the first image 21 and the first insertion vector V_{A1} becomes equal to the angle between a vector parallel to the direction of a predetermined landmark corresponding to another predetermined landmark of the second image 22 and the second insertion vector V_{A2} with a vector indicating the direction of another predetermined landmark as a basis, instead of the normal vector of the body surface S. For example, it is considered that a landmark with less fluctuation in the direction of the landmark according to the phase is used as the predetermined landmark. For example, a backbone may be used as a landmark, and the angle may be calculated based on the position of an N-th vertebra, (The center coordinates) of an organ, such as spleen or kidney, may be used as a basis.

“The angle between the direction of the predetermined landmark and the first insertion direction” means a smaller angle among the angles between the direction of the predetermined landmark and the first insertion direction, and “the angle between the direction of the predetermined landmark and the second insertion direction” means a smaller angle among the angles between the direction of the predetermined landmark and the second insertion direction.

The observation condition determination unit 13 determines a position separated at the distance D_{A1} between the first insertion position Q_{A1} and the first tip position P_{A1} in the direction of the second insertion vector V_{A2} from the second insertion position Q_{A2} as the second tip position P_{A2}. With the above, the observation condition determination unit 13 determines the second tip position P_{A2} such that θ_{A1}=θ_{A2} and D_{A1}=D_{A2} are established in FIG. 4.

The observation condition determination unit 13 determines the second tip position P_{A2}, the second insertion position Q_{A2}, the second insertion vector V_{A2}, and the second imaging direction in the second image 22 as the second observation condition. In the second observation condition, similarly to the first observation condition, it is assumed that other parameters necessary for generating an observation image from a three-dimensional image are set in advance according to the image angle, the focal distance, or the like of the virtual endoscope device M1.

As a method of determining the direction corresponding to the first insertion direction V_{A1} as the second insertion direction V_{A2}, the observation condition determination unit 13 may convert the tip portion of the virtual endoscope device in the first image to the coordinates in the second image and may align the coordinates with a vector from the insertion position toward the tip position after conversion in the second image. In this case, the observation condition determination unit 13 acquires the position corresponding to the first insertion position Q_{A1} as the second insertion position Q_{A2} based on the deformation information, acquires the position corresponding to the first tip position P_{A1} as the second tip position P_{A2}, and may determine the direction from the second insertion position Q_{A2} toward the second tip position P_{A2} as the second insertion vector V_{A2}. Similarly, the center-of-gravity position of the virtual endoscope device in the first image may be converted to the coordinates in the second image, and the coordinates may be set as a vector from the insertion position toward the tip position after conversion in the second image. In these cases, a second observation image 32 to be a virtual endoscopic image obtained by visualizing the inside of the subject is generated and displayed based on the second observation condition, whereby it is possible to provide useful information for easily and accurately determining whether or not the insertion position into the inside of the subject, the tip position, and the insertion direction are appropriate while making the first insertion direction V_{A1} from the first insertion position Q_{A1} toward the first tip position P_{A1} (the center-of-gravity position of the virtual endoscope device) correspond to the second insertion direction V_{A2} from the second insertion position Q_{A2} toward the second tip position P_{A2} (or the center-of-gravity position of the virtual endoscope device).

The image generation unit 14 generates the first observation image 31 to be a virtual endoscopic image obtained by visualizing the inside of the celom of the subject from the first image 21 based on the first observation condition, and generates the second observation image 32 to be a virtual endoscopic image obtained by visualizing the inside of the subject from the second image 22 based on the second observation condition. In the first observation condition and the second observation condition, the insertion positions Q_{A1} and Q_{A2} of the virtual endoscope device M1, the insertion depths of D_{A1} and D_{A2} from the insertion positions Q_{A1} and Q_{A2}, the insertion directions V_{A1} and V_{A2} from the insertion positions Q_{A1} and Q_{A2}, and the relative imaging directions with respect to the insertion directions V_{A1} and V_{A2} correspond to each other. For this reason, the first observation image 31 and the second observation image 32 show the inside of the subject in the phase corresponding to the first image 21 and the inside of the subject in the phase corresponding to the second image 22 in the substantially same composition while making the insertion positions Q_{A1} and Q_{A2} of the virtual endoscope device M1, the insertion depths D_{A1} and D_{A2} of the insertion positions Q_{A1} and Q_{A2}, and the insertion directions V_{A1} and V_{A2} from the insertion positions Q_{A1} and Q_{A2} correspond to each other, and are images in which the shape of an organ in the image is in a deformation state according to the phase corresponding to each of the first image 21 and the second image 22. The image generation unit 14 generates a desired image, such as volume rendering image, from the first image 21 or the second image 22 in a process of image processing of this embodiment as necessary.

The image generation unit 14 may acquire a deformed first image 21A obtained by deforming the first image 21 based on the deformation information, and may generate the second observation image 32 obtained by visualizing the inside of the subject based on the second observation condition using the second imaging direction as the camera posture with a second tip position P_{A2} in the deformed first image 21A as a viewpoint. Since the second image 22 and the deformed first image 21A have the pixels arranged at the same position, the observation image generated from the deformed first image 21A based on the second observation condition shows the inside of the subject having the same shape in the same composition with the observation image 32 generated from the second image 22 based on the second observation condition. For this reason, in all observation images generated from the second image 22 and the deformed first image 21A, the relative relationship of the insertion position, the tip position, the organ shape, and the like is the same, and can be used in order to confirm the inside of the subject in the phase corresponding to the second image 22, or the insertion position, the tip position, and the like of the virtual endoscopic image.

The output unit 15 outputs the images generated by the image generation unit 14 to the display device 3. The display device 3 displays the first observation image 31 and the second observation image 32 on the display screen in response to a request of the output unit 15. The output unit 15 may output the first observation image 31 and the second observation image 32 simultaneously, and may display the first observation image 31 and the second observation image 32 on the display screen of the display device 3 in parallel. Alternatively, the output unit 15 may selectively output the first observation image 31 and the second observation image 32, and may switch and display the first observation image 31 and the second observation image 32 on the display screen of the display device 3. The output unit 15 instructs the display device 3 to display desired information on the display screen in a process of image processing of this embodiment as necessary.

The determination unit 16 acquires a predetermined anatomical structure (for example, a blood vessel, a bone, or an organ, such as a lung) extracted from the second image 22 by an arbitrary method, and determines whether or not a line segment (line segment to be determined) connecting the second insertion position Q_{A2} and the second tip position P_{A2} is close at an unallowable distance or less to the anatomical structure included in the second image 22. The determination unit 16 extracts an overlap portion of the line segment to be determined and the anatomical structure in the second image 22 as a proximal portion which is close to the anatomical structure inside the subject at a predetermined allowable distance or less. In a case where the line segment to be determined and the anatomical structure in the second image 22 do not overlap each other, it is determined that there is no proximal portion. The line segment connecting the second insertion position Q_{A2} and the second tip position P_{A2} indicates a position where the rigid insertion portion of the medical instrument, such as a virtual endoscope device M1 or a virtual rigid treatment tool M2, is arranged. In order to secure safety of the inside of the subject, the rigid insertion portion should be arranged to be separated from an anatomical structure, such as a blood vessel, which is not a processing target, and it is preferable to confirm such that the line segment to be determined indicating the arrangement position of the rigid insertion portion is at an unallowable distance or less from the anatomical structure in a surgery simulation.

An arbitrary determination method can be applied as long as it is possible to determine whether or not the line segment to be determined is close at a predetermined distance or less to the anatomical structure included in the second image 22. For example, the shortest distance among the distances from respective pixels positioned in an organ may be calculated for each pixel positioned on the line segment to be determined, in a case where the calculated shortest distance is equal to or less than a predetermined threshold, it may be determined that the pixel is a proximal pixel, and a portion on the line segment to be determined having the determined proximal pixel may be extracted as a proximal portion to determine the presence or absence of a proximal portion.

In a case where the line segment to be determined has a proximal portion, the determination unit 16 instructs the output unit 15 to output warning display. If the instruction to output warning display is received from the determination unit 16, the output unit 15 acquires information for specifying the proximal portion from the determination unit 16, and outputs the instruction of warning display and information necessary for warning display to the display device 3. Then, the display device 3 acquires the proximal portion from the output unit 15, and performs warning display by color-coding and distinctively displaying the proximal portion in color coding according to a predetermined warning format.

The determination unit 16 can apply an index, such as an arrow, or an arbitrary method, such as bold-line display, for distinctive display of the proximal portion. The determination unit 16 can apply an arbitrary warning method in conjunction with distinctive display of the proximal portion or instead of distinctive display of the proximal portion. For example, the effect that the line segment to be determined and the anatomical structure are at a predetermined distance or less, such as “a proximal portion is present”, may be displayed in a dialogue box, an index indicating a warning may be shown, or an arbitrary warning display method may be applied. The determination unit 16 may perform a warning by warning sound, a voice message, or the like in conjunction with warning display or instead of warning display. The determination unit 16 may perform warning display automatically in a case where there is a proximal portion, or may output the determination result in response to a request from the user.

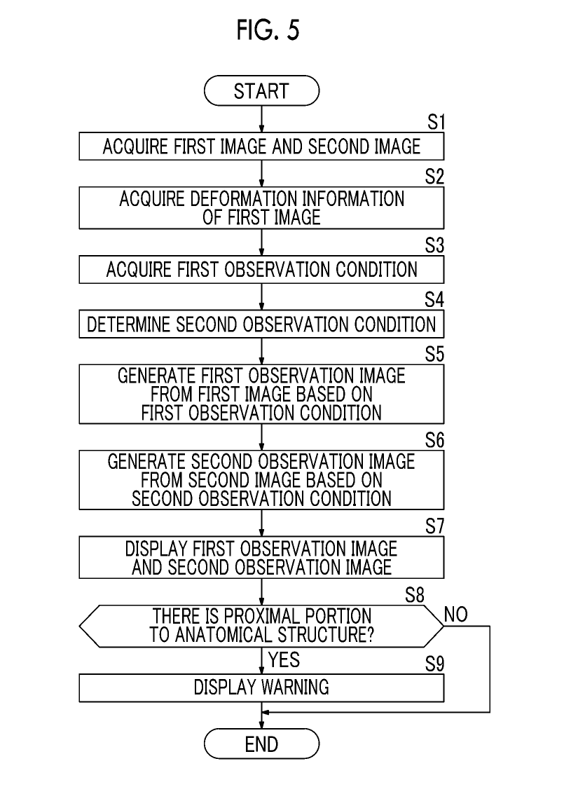

FIG. 5 is a flowchart showing an operation procedure of the image processing device 1. The image acquisition unit 11 acquires the first image 21 and the second image 22 (Step S1). The first image 21 and the second image 22 are two pieces of three-dimensional image data in an expiration phase and an inspiration phase.

The deformation information acquisition unit 12 performs image alignment on the first image 21 and the second image 22, and acquires the deformation information for deforming the first image 21 such that each pixel of the first image 21 is positioned at the position of each corresponding pixel of the second image 22 (Step S2).

As shown in FIGS. 2 and 3, the observation condition determination unit 13 acquires the first tip position P_{A1}, the first insertion position Q_{A1}, the first insertion vector V_{A1} from the first insertion position Q_{A1} toward the first tip position P_{A1}, and the first imaging direction with respect to the first insertion vector V_{A1} of the virtual endoscope device M1 as the first observation condition for the first image 21 based on the input of the position of the user from the display screen (Step S3).

The observation condition determination unit 13 specifies the second insertion position Q_{A2} to be the position corresponding to the first insertion position Q_{A1} in the second image 22 based on the first observation condition and the deformation information in the first image 21. The second tip position P_{A2} is specified such that the direction corresponding to the first insertion vector V_{A1} from the first insertion position Q_{A1} toward the first tip position P_{A1} becomes the second insertion vector V_{A2} from the second insertion position Q_{A2} toward the second tip position P_{A2} to be the position of the tip portion of the surgical instrument in the second image 22, and the distance D_{A1} between the first insertion position Q_{A1} and the first tip position P_{A1} becomes equal to the distance D_{A2} between the second insertion position Q_{A2} and the second tip position P_{A2}. As shown in FIG. 4, the second tip position P_{A2} is determined such that θ_{A1}=θ_{A2} and D_{A1}=D_{A2} are established. The second insertion position Q_{A2}, the second tip position P_{A2}, the second insertion vector V_{A2} from the second insertion position Q_{A2} toward the second tip position P_{A2}, and the second imaging direction with respect to the second insertion vector V_{A2} are determined as the second observation condition (Step S4).

The image generation unit 14 generates the first observation image 31 from the first image 21 based on the first observation condition using the first imaging direction as the camera posture with the first tip position P_{A1} as a viewpoint (Step S5), and generates the second observation image 32 from the second image 22 based on the second observation condition using the second imaging direction as the camera posture with the second tip position P_{A2} as a viewpoint (Step S6).

For example, the output unit 15 outputs the first observation image 31 generated in Step S5 and the second observation image 32 generated in Step S6 to the display device 3 simultaneously, and allows the first observation image 31 and the second observation image 32 to be displayed on the display surface simultaneously (Step S7).

Next, the determination unit 16 determines whether or not the line segment (line segment to be determined) connecting the second tip position P_{A2} and the second insertion position Q_{A2} is close at an unallowable distance or less to the anatomical structure included in the second image 22 or has a proximal portion. In a case where there is a proximal portion (Step S8, YES), the output unit 15 is instructed to perform warning display. Then, the output unit 15 outputs an instruction of warning display to the display device 3, and the display device 3 performs warning display by color-coding and distinctively displaying the proximal portion (Step S9).

According to this embodiment, in the second image 22 of the phase different from the first image 21, the tip position (second tip position P_{A2}) of the virtual medical instrument in the second image can be determined while making the insertion position (first insertion position Q_{A1}) and the insertion direction (first insertion direction V_{A1}) of the virtual endoscope device M1 as the virtual medical instrument in the first image 21 correspond to the insertion position (second insertion position Q_{A2}) and the insertion direction (second insertion direction V_{A2}) of the virtual medical instrument in the second image, and the second observation image 32 obtained by visualizing the inside of the subject with the second tip position P_{A2} as a viewpoint can be generated. For this reason, the inside of the subject shown in the first observation image 31 and the inside of the subject shown in the second observation image 32 are shown in a composition in which the insertion direction from the insertion port and the tip position are made correspondent, and the shape of the organ in both images is in the deformation state according to the phase corresponding to each of the first image 21 and the second image 22.

As in this embodiment, in the second image 22 of the phase different from the first image 21, in a case where the tip position (second tip position P_{A2}) of the virtual medical instrument in the second image is determined while making the insertion position (first insertion position Q_{A1}), the insertion direction (first insertion direction V_{A1}), the insertion depth (the distance D_{A1} between the first insertion position and the first viewpoint) of the virtual endoscope device M1 as a virtual medical instrument in the first image 21 correspond to the insertion position (second insertion position Q_{A2}), the insertion direction (second insertion direction V_{A2}), and the insertion depth (the distance D_{A2} between the second insertion position and the second viewpoint) of the virtual medical instrument in the second image, and the second observation image 32 obtained by visualizing the inside of the subject in the second imaging direction corresponding to the first imaging direction is generated with the second tip position P_{A2} as a viewpoint, the inside of the subject shown in the first observation image 31 and the inside of the subject shown in the second observation image 32 are shown in the same composition in which the insertion direction from the insertion port, the insertion depth, the tip position, and the imaging direction are made correspondent, and the shape of the organ in both shapes is in the deformation state according to the phase corresponding to each of the first image 21 and the second image 22.

Accordingly, in this embodiment, even in a case where there are a plurality of phases of respiration or pulsation causing deformation of an anatomical structure inside the subject in a period during which the medical instrument having the rigid insertion portion is arranged inside the subject, for example, during surgery, or the like, the generated second observation image 32 can be observed by the user, whereby it is possible to provide useful information for easily and accurately determining whether or not the insertion position into the inside of the subject, the tip position, and the insertion direction are appropriate when carrying out treatment or observation of the inside of the subject in the phase corresponding to the second image 22.

For example, the user compares the first observation image 31 with the second observation image 32, and in a case where the insertion position Q_{A1}, the insertion vector V_{A1}, and the insertion depth D_{A1} of the virtual endoscope device M1 set in the phase corresponding to the first image 21 are maintained, it is possible to confirm how the observation image changes in the phase corresponding to the second image 22 by deforming the inside of the subject according to the phase. Instead of displaying the two observation images 31 and 32, the first observation image 31 may not be generated, and only the second observation image 32 may be displayed on the display surface. In this case, the user can confirm how the observation image changes by deforming the inside of the subject in the second observation image in the phase different from the first image 21. In a case where the first observation image 31 and the second observation image 32 corresponding to different respiration phases or pulsation phases are generated and displayed, even if deformation of an organ or the like inside the subject occurs due to respiration of the subject during surgery or the like, it is possible to provide useful information for easily and accurately determining whether or not the insertion position into the inside of the subject, the tip position, and the insertion direction are appropriate in both different phases. Even in a case where the first image and the second image represent the subject in different postures, the first observation image 31 and the second observation image 32 corresponding to the subject in different postures are generated and displayed, whereby it is possible to provide useful information for easily and accurately determining whether or not the insertion position into the inside of the subject, the tip position, and the insertion direction are appropriate in different postures even if deformation of an organ or the like inside the subject occurs due to the difference in posture.

As described above, in a case where the second insertion direction V_{A2} is determined such that the angle of the normal vector T_{A1} of the body surface S and the first insertion vector V_{A1} of the first image 21 at the first insertion position Q_{A1} becomes equal to the angle between the normal vector T_{A2} of the body surface S and the second insertion direction V_{A2} of the second image 22 at the second insertion position Q_{A2}, it is possible to determine the second observation condition such that the insertion angle with respect to the body surface S of the subject at the insertion position of the virtual endoscope device M1 is equal in different phases. For this reason, the generated second observation image is used as a reference, whereby it is possible to observe a state of the inside of the celom in the phase corresponding to the first image 21 and the phase corresponding to the second image 22 in a case where the insertion angle with respect to the body surface S of the subject is made coincident.

As described above, in a case where the determination unit 16 which determines whether or not the line segment (a portion corresponding to the rigid insertion portion of the medical instrument, such as a virtual endoscope device) connecting the second tip position P_{A2} and the second insertion position Q_{A2} is close at an unallowable distance or less to the anatomical structure included in the second image 22 is provided, it is possible to provide useful information for determining whether or not the arrangement of the rigid insertion portion of the medical instrument or the insertion path is appropriate in a surgery simulation or the like. The output unit 15 outputs a warning, such as warning display or warning sound, in a case where there is a proximal portion, whereby it is possible to appropriately call user's attention. In a case where the proximal portion is distinctively displayed, whereby it is possible to allow the user to easily and accurately understand the presence and the position of the proximal portion.

In endoscopic surgery using a plurality of medical instruments, there is a case where desired treatment is carried out using a rigid treatment tool, such as a scalpel or a needle, while observing a treatment part with a rigid endoscope device. In this case, an insertion port is provided in each of the rigid endoscope device and the rigid treatment tool, and a desired surgical instrument is inserted from each insertion port to an appropriate position to carry out desired observation and treatment. For this reason, as a second embodiment which is a modification of the above-described first embodiment, it is preferable that, in a case where a plurality of different first observation conditions are set in the first image 21, the observation condition determination unit 13 determines a plurality of second observation conditions corresponding to a plurality of first observation conditions. Hereinafter, the second embodiment will be described.

The second embodiment is different from the first embodiment in that, in a case where a plurality of different first observation conditions are set in the first image 21, the observation condition determination unit 13 determines a plurality of second observation conditions corresponding to a plurality of first observation conditions, the image generation unit 14 generates a plurality of first observation images 31 corresponding to a plurality of first observation conditions and a plurality of second observation images 32 corresponding to a plurality of second observation conditions, a plurality of first observation images 31 and a plurality of second observation images 32 generated by the output unit 15 are output to the display device 3, and the display device 3 displays a plurality of first observation images 31 and a plurality of second observation images 32. Except for these differences, the basic functions or configurations of the respective units of the image processing device 1 are common, and the flow of image processing shown in FIG. 5 is common; thus, the flow of processing of the second embodiment will be described referring to FIG. 5, description of the common configurations, functions, and processing of the respective units of the second embodiment and the first embodiment will not be repeated, and description will be provided focusing on the different parts between the second embodiment and the first embodiment.

In the second embodiment, the acquisition processing of the first image 21 and the second image 22 (S1 of FIG. 5) and the deformation information acquisition processing (S2 of FIG. 5) are common to the first embodiment. In regard to the process of S3 shown in FIG. 5, as in the first embodiment, the observation condition determination unit 13 in the second embodiment acquires a plurality of first observation conditions according to user input.

In regard to the processing of S4 shown in FIG. 5, the observation condition determination unit 13 in the second embodiment acquires a plurality of first observation conditions, and as in the first embodiment, determines the second observation conditions corresponding to the respective first observation conditions. FIG. 4 shows an example where two different first observation conditions are set. For example, it can be considered that reference numeral M1 indicates a virtual endoscope device, and reference numeral M2 indicates another treatment tool, such as a scalpel. Description will be provided referring to FIG. 4. As in the first embodiment, the observation condition determination unit 13 determines the second insertion position Q_{A2} and the second tip position P_{A2} in the second image 22 based on the first insertion position Q_{A1} and the first tip position P_{A1} in the first image 21, and in regard to the first insertion position Q_{B1} and the first tip position P_{B1}, determines a second insertion position Q_{B2} and a second tip position P_{B2} in the second image 22 based on the first insertion position Q_{B1} and the first tip position P_{B1} in the first image 21 as in first embodiment.

In detail, the observation condition determination unit 13 specifies the second insertion position Q_{B2} of the second image 22 corresponding to the first insertion position Q_{B1}, and acquires the normal vector T_{B1} of the body surface S at the first insertion position Q_{B1} and the normal vector T_{B2} of the body surface S at the second insertion position Q_{B2}. The second insertion vector V_{B2} is determined such that the angle θ_{B2} between the second insertion vector V_{B2} and the normal vector T_{B2} in the second image 22 becomes equal to the angle θ_{B1} between the first insertion vector V_{B1} and the normal vector T_{B1} in the first image 21. A position separated from the second insertion position Q_{B2} at the distance D_{B1} between the first insertion position Q_{B1} and the first tip position P_{B1} in the direction of the second insertion vector V_{B2} is determined as the second tip position P_{B2}. As in the first embodiment, the observation condition determination unit 13 determines the second imaging direction such that the relative relationship of the first imaging direction with respect to the first insertion vector V_{A1} becomes equal to the relative relationship of the second imaging direction with respect to the second insertion vector V_{A2}. With the above, in FIG. 4, the observation condition determination unit 13 determines the second tip position P_{B2} such that θ_{B1}=θ_{B2} and D_{B1}=D_{B2} are established. Even in a case where there are more first observation conditions, the observation condition determination unit 13 determines the corresponding second observation conditions similarly.

In regard to the processing of S5 shown in FIG. 5, the image generation unit 14 in the second embodiment generates a plurality of first observation images corresponding to a plurality of first observation conditions from the first image 21. In regard to the processing of S6 shown in FIG. 5, the image generation unit 14 in the second embodiment generates a plurality of second observation images (images generated from the second image 22 or images generated from the deformed first image 21A) corresponding to a plurality of second observation conditions. In regard to the processing of S7 shown in FIG. 5, the output unit 15 outputs the generated second observation images corresponding to a plurality of second observation conditions to the display device 3 to display the second observation images on the display screen. The image generation unit 14 and the output unit may perform image generation processing and image output processing for all of a plurality of first observation images 31 and a plurality of second observation images 32, or may perform image generation processing and image output processing only for a part of a plurality of first observation images 31 and a plurality of second observation images 32.

In regard to the processing of S8 and S9 shown in FIG. 5, the determination unit 16 in the second embodiment determines whether or not the line segment (line segment to be determined) connecting the second insertion position and the second tip position is at a predetermined distance or less from the anatomical structure included in the subject for each of a plurality of second observation conditions, and in a case where there is a proximal portion among a plurality of line segments to be determined (S8 of FIG. 5, YES), performs warning display by color-coding and distinctively displaying the proximal portion (S9 of FIG. 5). In this case, it is possible to easily and efficiently understand whether or not a surgical instrument inserted into each of a plurality of insertion positions is arranged to be appropriately separated from an organ. The determination unit 16 may perform warning display only for a part of a plurality of line segments to be determined, or may not perform warning display.

As in the second embodiment, a plurality of generated second observation images corresponding to a plurality of second observation conditions are output to the display device 3 and displayed on the display screen, whereby it is possible to easily and efficiently understand whether or not a plurality of insertion positions corresponding to a plurality of medical instruments having a rigid insertion portion and the insertion depths or the insertion directions from the insertion positions are set even in a case where there is deformation of the inside of the object according to the phases of the first image 21 and the second image 22. In endoscopic surgery, in order to observe a plurality of treatment parts or one treatment part at a plurality of angles with a rigid endoscope device according to treatment purposes or treatment methods of surgery, there is a case where the rigid endoscope device is inserted into a plurality of insertion ports to observe a treatment part. In this case, a plurality of second observation images are referred to, whereby it is possible to confirm the distance from a processing target, an observation range, or the like while corresponding a plurality of insertion positions and the insertion depths from the insertion positions of the rigid endoscope device or the insertion directions to a plurality of insertion ports.

In the second embodiment, the image generation unit 14 may further generate another pseudo three-dimensional image representing the subject from the second image 22 or the deformed first image 21A such that a plurality of second insertion positions and a plurality of second tip positions corresponding to a plurality of second observation conditions are visible, and the output unit 15 may output the generated pseudo three-dimensional images to the display device 3 to display the pseudo three-dimensional images on the display screen. Physicians observe the pseudo three-dimensional images representing the subject such that a plurality of second insertion positions and a plurality of second tip positions corresponding to a plurality of second observation condition are visible in the phase corresponding to the second image 22, thereby easily understanding the deformation state of the inside of the subject in the phase corresponding to the second image 22 and the relative arrangement of the surgical instruments having the rigid insertion portion corresponding to a plurality of second observation conditions and obtaining effective information for easily and efficiently determining whether or not a plurality of insertion positions and the insertion depths from the insertion positions or the insertion directions are arranged in appropriate positions and directions.

The number of images input to the image processing device 1 is not limited to two, and three or more images may be input to the image processing device 1. For example, in a case where three images (first to third images) are input to the image processing device 1, the image acquisition unit 11 acquires the first to third images, and the deformation information acquisition unit 12 may perform alignment in the first image and the second image, and may perform alignment in the first image and the third image. The observation condition determination unit 13 may determine a second observation condition (a second tip position corresponding to a first tip position and a second insertion position corresponding to a first insertion position) and a third observation condition (a third tip position corresponding to a first tip position and a third insertion position corresponding to a first insertion position) corresponding to the first observation condition set in the first image in both of the second image and the third image. The image generation unit 14 may generate a second observation image based on the second observation condition from the second image, and may generate a third observation image based on the third observation condition from the third image. The output unit 15 may output the second observation image and the third observation image to the display device 3. The determination unit 16 may determine whether or not a line segment connecting the second tip position and the second insertion position is at a distance equal to or less than a predetermined threshold from the anatomical structure of the subject based on the second observation condition (the second tip position corresponding to the first tip position and the second insertion position corresponding to the first insertion position), and may determine whether or not a line segment to be determined connecting the third tip position and the third insertion position is at a distance equal to or less than a predetermined threshold from the anatomical structure of the subject based on the third observation condition (the third tip position corresponding to the first tip position and the third insertion position corresponding to the first insertion position).

In the respective embodiments described above, the processing sequence of the deformation information acquisition processing (S2) and the first observation condition acquisition processing (S3) may be changed. In the respective embodiments, the processing of S8 and S9 may be omitted, and the image processing device 1 may not include the determination unit 16. The first observation image generation processing (S5) may be carried out at an arbitrary timing after the first observation condition acquisition processing (S3) and before the first observation image display processing (S7), or the first observation image generation processing (S5) and the first observation image display processing may be omitted.

Although the invention has been described based on the preferred embodiments, the image processing device, the method, and the program of the invention are not limited to the above-described embodiments, and various alterations and modifications formed from the configurations of the above-described embodiments are also included in the scope of the invention.

What is claimed is:

1. An image processing device comprising:
a three-dimensional image acquisition unit which acquires a first image and a second image respectively representing the inside of a subject in different phases as three-dimensional images captured using a medical imaging device;
a deformation information acquisition unit which acquires deformation information for deforming the first image such that corresponding positions of the first image and the second image are aligned with each other;
an observation condition determination unit which acquires a first insertion position to be the insertion position of a surgical instrument having an elongated rigid insertion portion inserted into the body of the subject and a first tip position to be the position of a tip portion of the surgical instrument from the first image as a first observation condition, based on the first observation condition and the deformation information, specifies a second insertion position to be the position on the second image corresponding to the first insertion position and specifies a second tip position such that a direction corresponding to a first insertion direction from the first insertion position toward the first tip position becomes a second insertion direction from the second insertion position toward the second tip position to be the position of the tip portion of the surgical instrument in the second image, and determines the second insertion position and the second tip position as a second observation condition; and
an image generation unit which generates a second observation image obtained by visualizing the inside of the subject from the second tip position from a deformed first image obtained by deforming the first image based on the deformation information or the second image based on the second observation condition with the second tip position as a viewpoint.

2. The image processing device according to claim 1,
wherein the surgical instrument is an endoscope device,
the observation condition determination unit specifies the second imaging direction such that the relative relationship between the first insertion direction in the first image and a first imaging direction to be the imaging direction of the endoscope device becomes equal to the relative relationship between the second insertion direction in the second image and a second imaging direction to be the imaging direction of the endoscope device, and
the image generation unit generates the second observation image by visualizing the inside of the subject in the second imaging direction from the second tip position.

3. The image processing device according to claim 1,
wherein the observation condition determination unit specifies the second tip position such that the distance between the first insertion position and the first tip position becomes equal to the distance between the second insertion position and the second tip position, and determines the second insertion position and the second tip position as the second observation condition.

4. The image processing device according to claim 3,
wherein the observation condition determination unit specifies the second insertion direction such that the direction corresponding to the first insertion direction becomes the second insertion direction by specifying the second insertion direction such that the angle between the direction of a predetermined landmark included in the first image and the first insertion direction becomes equal to the angle between the direction of the predetermined landmark included in the second image and the second insertion direction.

5. The image processing device according to claim 1,
wherein the observation condition determination unit specifies the position on the second image corresponding to the first tip position as the second tip position, and determines the second insertion position and the second tip position as the second observation condition.

6. The image processing device according to claim 1,
wherein the observation condition determination unit acquires a plurality of first observation conditions from the first image and determines a plurality of second observation conditions corresponding to the plurality of first observation conditions based on the plurality of first observation conditions and the deformation information.

7. The image processing device according to claim 1,
wherein the first image and the second image respectively represent the subject in an expiration phase and an inspiration phase.

8. The image processing device according to claim 1,
wherein the first image and the second image respectively represent the subject in different pulsation phases.

9. The image processing device according to claim 1,
wherein the first image and the second image represent the subject in different postures.

10. The image processing device according to claim 1, further comprising:
a determination unit which determines whether or not a line segment connecting the second insertion position and the second tip position is equal to or less than a predetermined distance from an anatomical structure included in the second image.

11. A method of operating an image processing device, the method comprising:
a three-dimensional image acquisition step of acquiring a first image and a second image respectively representing the inside of a subject in different phases as three-dimensional images captured using a medical imaging device;
a deformation information acquisition step of acquiring deformation information for deforming the first image such that corresponding positions of the first image and the second image are aligned with each other;
an observation condition determination step of acquiring a first insertion position to be the insertion position of a surgical instrument having an elongated rigid insertion portion inserted into the body of the subject and a first tip position to be the position of a tip portion of the surgical instrument from the first image as a first observation condition, based on the first observation condition and the deformation information, specifying a second insertion position to be the position on the second image corresponding to the first insertion position and specifying a second tip position such that a direction corresponding to a first insertion direction from the first insertion position toward the first tip position becomes a second insertion direction from the second insertion position toward the second tip position to be the position of the tip portion of the surgical instrument in the second image, and determining the second insertion position and the second tip position as a second observation condition; and
an image generation step of generating a second observation image obtained by visualizing the inside of the subject from the second tip position from a deformed first image obtained by deforming the first image based on the deformation information or the second image based on the second observation condition with the second tip position as a viewpoint.

12. A non transitory computer readable medium having an image processing program stored therein, which causes a computer to execute:
a three-dimensional image acquisition step of acquiring a first image and a second image respectively representing the inside of a subject in different phases as three-dimensional images captured using a medical imaging device;
a deformation information acquisition step of acquiring deformation information for deforming the first image such that corresponding positions of the first image and the second image are aligned with each other;
an observation condition determination step of acquiring a first insertion position to be the insertion position of a surgical instrument having an elongated rigid insertion portion inserted into the body of the subject and a first tip position to be the position of a tip portion of the surgical instrument from the first image as a first observation condition, based on the first observation condition and the deformation information, specifying a second insertion position to be the position on the second image corresponding to the first insertion position and specifying a second tip position such that a direction corresponding to a first insertion direction from the first insertion position toward the first tip position becomes a second insertion direction from the second insertion position toward the second tip position to be the position of the tip portion of the surgical instrument in the second image, and determining the second insertion position and the second tip position as a second observation condition; and
an image generation step of generating a second observation image obtained by visualizing the inside of the subject from the second tip position from a deformed first image obtained by deforming the first image based on the deformation information or the second image based on the second observation condition with the second tip position as a viewpoint.
